(19) United States
(12) Patent Application Publication
(10) Pub. No.: US 2003/0059809 A1
(43) Pub. Date: Mar. 27, 2003

(54) Biochips,preparation and uses

(76) Inventors: Todor Vujasinovic, Paris (FR); Sylvie Dumas, Paris (FR); Jacques Mallet, Paris (FR)

Correspondence Address: NIXON & VANDERHYE, PC, 1100 N GLEBE ROAD, 8TH FLOOR, ARLINGTON, VA, 22201-4714, US

(21) Appl. No.: 10/168,143

(22) PCT Filed: Dec. 18, 2000
(86) PCT No.: PCT/FR00/03572

(51) Int. Cl. C12Q 1/68; C12M 1/34

(52) U.S. Cl. 435/6; 435/287.2

(57) ABSTRACT

The present invention concerns micro-arrays, their preparation and their uses. In particular, it concerns micro-arrays composed of nucleic acids immobilised on a support by means of arms in arborescent form and/or arms carrying a negative charge and/or directly on the supports carrying a negative charge. The methods and micro-arrays according to the present invention can be used for genetic expression detection or analysis, for research of genes of interest, or for diagnostic applications, for example.

The present invention relates to the field of biology and genetics. In particular, it concerns new compositions and methods for the preparation of nucleic acid micro-arrays and their uses. It deals notably with nucleic acid micro-arrays comprising particular nucleic acid populations and/or prepared from particular anchoring molecules or particular supports. The methods and nucleic acid micro-arrays according to the present invention can be used for detecting or analysing genetic expression, for searching for genes of interest, or for diagnostic applications, for example.

The “DNA micro-arrays” or, more commonly, nucleic acid micro-arrays, are miniaturized systems for genetic analysis on a large scale, enableing the study, for example, of the transcriptional activity of a large number of genes (genetic expression analysis), and the determination of the sequence of a large number of DNA fragments (including genetic polymorphism analyses), etc. Their general principle consists in:

fixing nucleic acid fragments on a support in an organised way, and in a miniaturized manner so that it is possible to attach a large number of different fragments on a reduced surface. The nucleic acid micro-array is the assembly composed of the support and the nucleic acid fragments attached to this support. In the present invention, the nucleic acids fixed on the micro-array are identified by the term “targets”. Each nucleic acid (or group of nucleic acids) that corresponds to a specific DNA or RNA sequence, is fixed to a determined position on the support (“spot”). Preferably, these micro-arrays have a density over 500 spots per cm², preferably over 1000 spots per cm² and even more preferably, over 5000 spots per cm².

Hybridising on the micro-array a population of nucleic acids for analysis (no matter what their nature or biological origin). Here we have called the nucleic acids for analysis by the term “probes”. During hybridisation, the nucleic acids present in the probe will attach themselves in a specific manner to the targets present on the micro-array, whose nucleic sequence is similar to all or part of the sequence of these probes.

Measuring the quantity of specifically hybridised probes on each of the micro-array targets. This measurement can be realized either by previous fluorescent or radioactive marking of the probes and the reading of the quantity of markings present after hybridisation on each target, or by using other measuring methods of the quantity of probe-target hybridisation for each target (such as, for example, and in non-exhaustive manner, the measurement of micro-currents induced through the formation of a double strand target-probe electric capacitance, or the direct measurement of the molecular mass of the probe fixed on each target).

A certain number of nucleic acid micro-arrays have been described in prior art. However, these nucleic acid micro-arrays present certain problems or limitations notably connected with the nature of the nucleic acid targets employed and/or the micro-array preparation conditions, that make their production and use difficult.

Therefore, the technology of single strand nucleic acid micro-arrays developed by the Affymetrix company consists in synthesizing oligonucleotides directly on the micro-array support by photo-lithography and DNA synthesis in solid phase. This principle, that can be obtained using other synthesis methods, is commonly identified by the expression “in situ synthesis”. However, till now, this principle has not permitted the synthesis of oligonucleotides more than 25 bases long with sufficient efficiency, and the use of these oligonucleotides (25 bases long or less) provokes the presence of numerous non-specific hybridisations of the probes, and consequently, unreliable experiment reproducibility when using this system.

With regard to this problem, it is well known that the specificity of hybridisation between two nucleic acid fragments (for example the probe-target complex for the nucleic acid micro-arrays) depends on the conditions in which the hybridisation is performed, the base compositions of these nucleic acids, their sequence similarity or identity, and the length of their identical sequence. Therefore, the longer the identical sequence between two fragments is, the more specific is the hybridisation. However, once a certain length has been exceeded, variable according to the sequences, but generally of the order of over 1000 bases, the secondary molecular structures can hinder the performance of the hybridisation reaction (folding in of the molecules on themselves, or even hybridisation of the molecules on themselves).

Therefore it would be useful to find technologies that permit the production of micro-arrays composed of all types of nucleic acids, in conditions that are compatible with efficient and sensitive hybridisation reading. However, the attachment of nucleic acids on micro-array supports still remains a problem to this day, notably because of the steric space occupied and the current physical and chemical modes of attachment on the supports.

The question of steric space occupation connected with the deposit of a large number of molecules on a very small surface (“spot” of each deposit on the micro-array) is an important one. In fact, it is essential that the deposited molecules (targets) be in sufficient number and density to permit the hybridisation measurement on the latter of the nucleic acid molecules to be analysed (probes). However, this density must not be so great that it hinders the access of the probe molecules to the target molecules because of the occupied space of the targets.

Moreover, the attachment conditions of the targets on the nucleic acid micro-array are just as important. In fact, this fixation must be stable, ideally of covalent type, but without diminishing the hybridisation capacity of the targets on the probes. From this point of view, the ideal situation would be that targets attachment be made by one of the ends of the target (5′ or 3′) and not on their length. Lastly, as far as the occupied steric space and the availability of targets along their whole length is concerned, but also including technical feasibility, the question has not yet been decided whether it is better to fix the targets directly on the micro-array support, or whether it is better to fix them through an intermediate “arm” (polymer molecule, whatever its nature or size).

The present invention now introduces advantageous solutions to the problems and limits concerning prior art techniques and products. These solutions are directed notably at the target attachment conditions on the support, as well as at the nature and/or the physical and chemical characteristics of the fixing molecules employed. The invention also describes the micro-arrays on which particular populations of target molecules are deposited, notably the target nucleic acid populations of established size.

The present invention describes notably a new approach for fixing target nucleic acids on micro-array supports. It can be set up with all types of target nucleic acids as defined below, and with all types of support, as defined below. Moreover, it can be set up just as well with target nucleic acids synthesized directly on the micro-array support (synthesis in situ), or synthesized independently, then fixed on the micro-array at another moment. Moreover, it can also be set up with molecules of interest other than polynucleotides.

More particularly, according to an initial aspect, in order to fix the targets on the micro-array, the present invention concerns the use of arms (or “linker” molecules) with a spatial structure (primary or secondary molecule structure) in arborescent form. In particular, the invention, in general, deals with the use of any molecule whose spatial structure is organised according to an arborescence principle, whether it has one or several degrees of liberty, such as target fixation “arms ” on a micro-array support.

Therefore, a first aim of the present invention concerns a micro-array characterised in that it comprises nucleic acids immobilised on a support by means of arms in arborescent form.

Another aspect of the present invention concerns a micro-array composed of single strand nucleic acids fixed on a support, in which the single strand nucleic acids have a length between approximately 25 and 100 nucleotides, preferably between approximately 30 and 60 nucleotides.

Another aspect of the invention also concerns a micro-array comprising immobilized nucleic acids on a support carrying a uniform negative electrical charge, or by means of arms carrying a negative electrical charge.

The invention also concerns the preparation and use of the micro-arrays as defined above, for genetic analysis, sequencing, genes research, diagnosis, etc.

To permit better understanding of the present invention, the definitions listed below have been provided. Except where specially indicated, the other technical terms employed in the present patent application should be interpreted according to their usual meaning.

Micro-array: according to the invention, a micro-array indicates any support on which target nucleic acids are deposited. Generally, the nucleic acids are immobilised on the support, preferably by covalent bonding. They can be immobilised directly on the support, or in an indirect manner, through the intermediate use of “arms”. This can involve micro-arrays for which the targets have been produced beforehand and then fixed on the support, or for which the targets are synthesized directly on the support (including photolithography and DNA synthesis in solid phase). The micro-arrays of the invention can include a low or very high number of targets, and the size of the total support surface occupied by the targets can vary.

In general, the term “arm” (or “spacer” or “linker”) indicates any molecule that can be used to fix target nucleic acids to a support. This relates to molecules that are not capable of forming hybrids, in a specific way, with probe nucleic acids. Therefore this deals preferably with molecules of an essentially non nucleic nature. In the constitution of the micro-arrays of the invention, only one type of “arm” or “spacer, or “linker”, or mixtures of differently structured “arms”, can be used by a micro-array. However, generally it is preferable to use a single type of arm on a single array in order to obtain a homogeneous signal. As an example, the arm molecule can be an organic molecule of a proteinic, glucidic or lipidic nature, or a non-organic synthesized polymer.

In the context of the present invention, the “target” nucleic acids present on the micro-array support can be of different nature and origin. Therefore, they can be single or double strand nucleic acids of natural, synthetic and/or semi-synthetic origin. In particular, they can be synthetic oligonucleotides, PCR products, genes or gene fragments, plasmids, single or double strand cDNA, RNA, etc. They can be nucleic acid populations isolated from biological samples, such as biopsies, samples of cells, mammal tissue or organs, notably human, samples of vegetable, animal, viral or bacterial origin, etc. Since this involves oligonucleotides, they are defined more particularly as single strand nucleic acid fragments with a length between 25 and 100 nucleotides, whether of DNA or RNA nature.

The micro-array support can vary in nature. Therefore, it can involve any support capable of receiving target nucleic acids, either directly or indirectly. In particular, the support can be composed of a flat and/or convex surface, and be of a solid or semi-solid nature. In addition, the support can be of variable shape and size. For instance, there exist supports that are circular, rectangular, square, etc. The support surface is preferably between 300 and 3000 mm², preferably between 400 and 1800 mm². For example, materials that can be used as supports include glass, silica, (notably vitreous silica after ammonia de-protection treatment), poly-lysine, amino-silanes and/or amino-reactive silanes.

The figures enclosed demonstrate various invention methods and describe more particularly:

FIG. No. 1: Use of fixing arms for arborescent targets; Use of non-arborescent fixing arms for targets;

FIG. No. 2: Uridine diphosphate glucose (UDPGluc) according to Harper Biochimie, R. K. Murray, D. K. Granner, P. A. Mayes, V. W. Rodwell, Publisher Mc Graw-Hill International (UK) Ltd.

FIG. No. 3: N bond within a glycoprotein.

FIG. No. 4: O bond within a glycoprotein.

FIG. No. 5: Capture probe anchorage on the micro-array by means of glycogen molecules (scale not respected).

FIG. No. 6: Capture probe anchorage on the micro-array by means of glycogen molecules (scale not respected).

FIG. No. 7: Glycogen molecule structure in a branching point according to Harper Biochimie.

FIG. No. 8: Amylopectin showing branching 1→6, according to Harper Biochimie.

FIG. No. 9: Diagram showing the agrecane of bovine nasal cartilage according to Harper Biochimie.

FIG. No. 10: Diagram showing human immunoglobulin polymers. The polypeptidic chains are represented by thick lines; the disulphide bridges that link the different polypeptidic chains are represented by thin lines.

As described above, a first object of the present invention relates to a micro-array characterised in that it comprises nucleic acids immobilized on a support by means of arms in arborescent form.

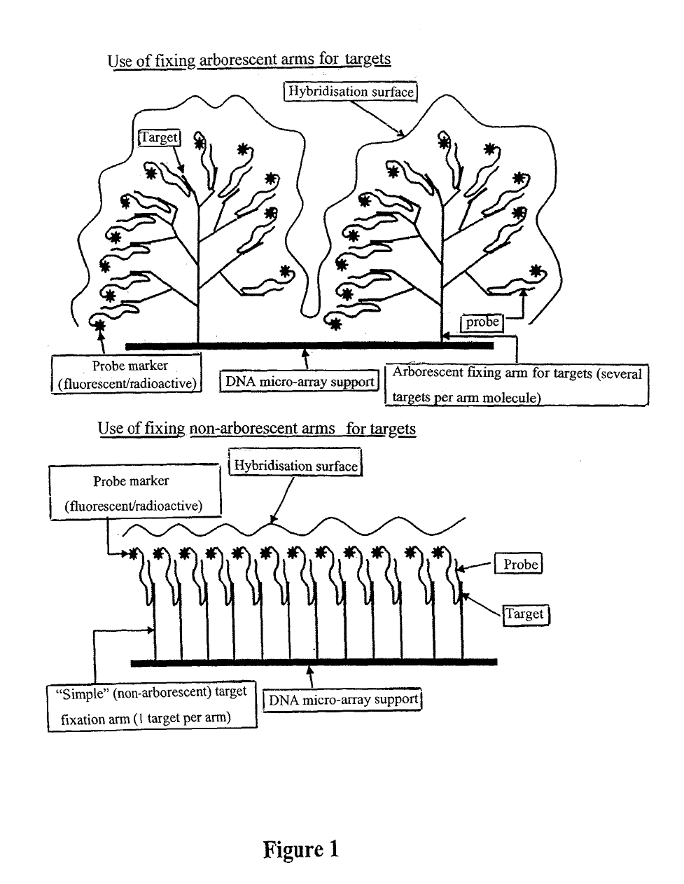

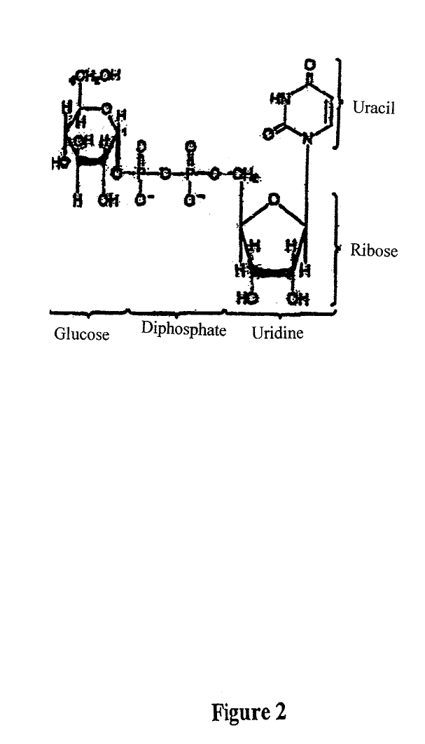

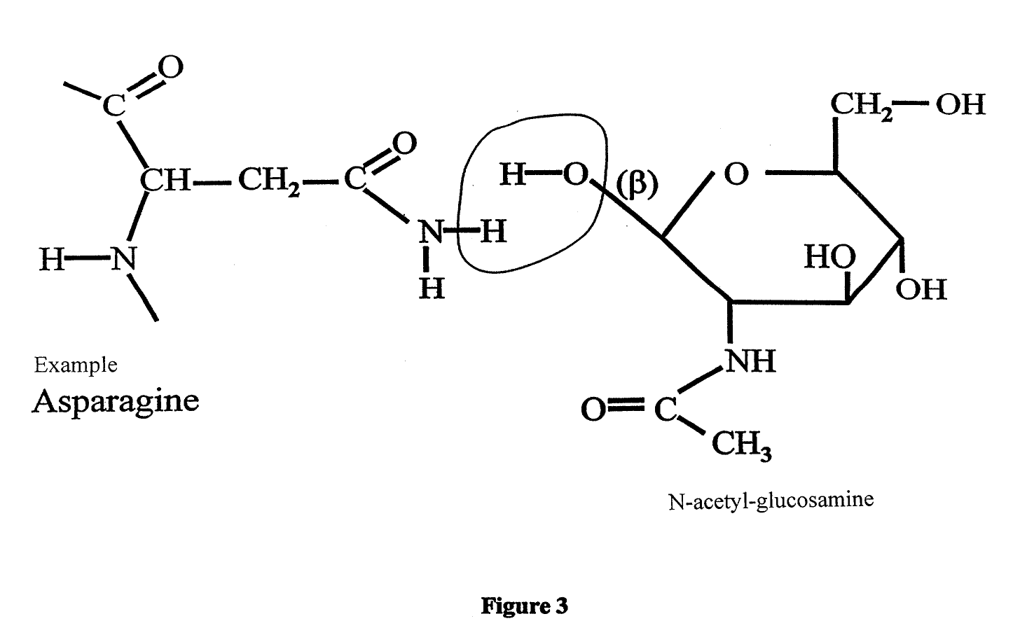

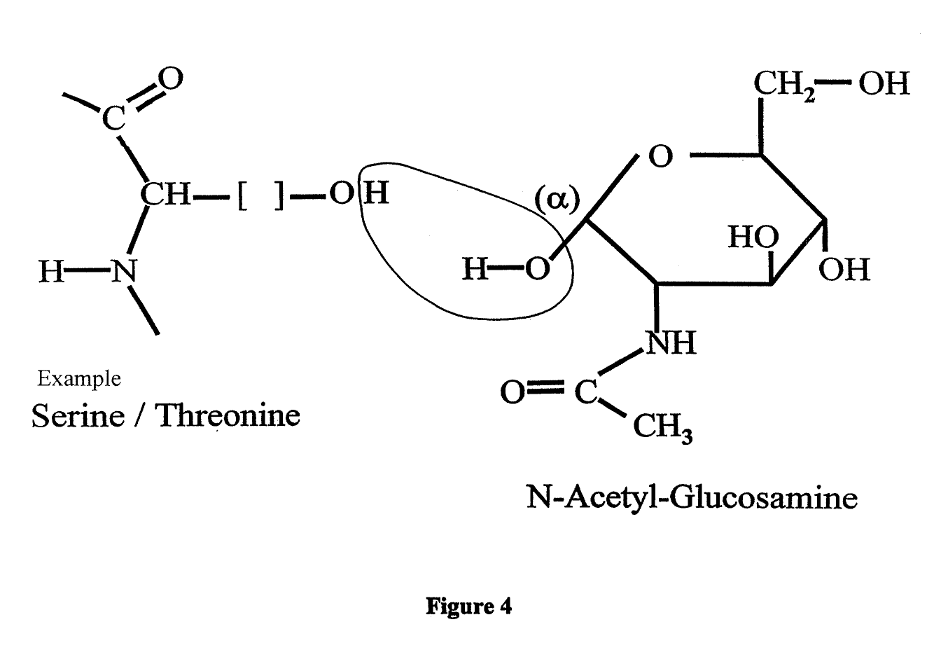

This principle, which to our knowledge is new, presents the advantage of being able to limit the difficulties connected with steric occupied space of molecules. Indeed, fixing target nucleic acids at the tips of the arborescence of such “arms”, provides an increased presentation surface of these targets to the probe nucleic acids under observation (See FIG. 1). In addition, it permits the reduction of the number of molecules to be fixed directly on the DNA micro-array supports (only the trunks of these “arms” are fixed). This makes it possible to obtain strong target nucleic acids density on each “spot” of the micro-array, while obtaining a reduced targets steric occupied space compared to situations where the targets are either fixed directly to the support, or fixed by means of a linear “arm”.

Arms of several physical natures have been proposed in prior art for fixing oligonucleotides onto supports. For instance, one can mention as a non-limiting example, the linear oligo-ethylene glycols between 26 and 105 atoms in length, selected for their capacity for covalent bonding to one of the ends of the oligonucleotide on one hand, and on the other hand, for their capacity to be fixed in covalent manner to various array supports. However, up till now, all the arms that have been proposed present a linear primary structure and/or are of synthetic nature (see WO00/43539 and WO99/10362). The present invention demonstrates that non-linear arms can be used for fixing nucleic acids to supports, especially arborescent shaped arms, preferably based on a polymer of biological origin.

In a more particular manner, the arm consists of a polymer with an arborescent spatial organisation, preferably oblong in shape. Advantageously, the polymer is a branched polymer with one or several branching levels.

The arborescent arm can be prepared from different organic molecules, especially organic polymers (that exist in nature), their derivatives, or mixed compounds, composed of an organic part and a synthetic part.

In a particular embodiment, the arm according to the invention comprises an organic polymer of biological origin. The biological compound used for the preparation of the arm can be a sugar, a polypeptide, a glycoprotein, a glycopolypeptide, an immunoglobulin, etc., even in isolated, compounded or multimerised form, or if necessary, functionalised or modified, notably by means of synthetic molecules or polymers, or reactive chemical groups.

Since this involves organic compounds that exist in nature, these can be purified from biological extracts or artificially synthesized extracts. According to a first advantageous embodiment, the organic polymer is a sugar or polysaccharide polymer.

The use of such sugar polymers for micro-array preparation, or more generally, the immobilization of nucleic acids, presents a wide range of interests and advantages, notably because of their chemical properties and their primary and secondary structure.

Thus, the polysaccharides are hydrophilic, which is a favourable condition for probe hybridisation on targets, and notably, for probes of a nucleotide nature (DNA, RNA) or derivatives (PNA). Indeed, the latter are hydrophilic and their hybridisation is performed in aqueous solution. A hydrophobic anchorage polymer would risk hindering hybridisation because of the repulsive effect on the hybridisation solution.

Moreover, polysaccharides, notably those described, have radicals that permit the creation of covalent bonds at the ends of the chain (all ends) because of the presence of hydroxyl functions (OH). The covalent bonds for example, can be phosphate bonds (C—P—C or C—P—P—C or C—Pn—C) such as those which exist in nucleotidic sugars or in sugar donor polynucleotides that intervene in the synthesis of polysaccharides and glycoproteins. A possible anchorage example is based on the steps of natural glycogen synthesis and notably the reaction of glucose-1-phosphate with uridine triphosphate (UTP) in the presence of the UDPGluc-pyrophosphorylase enzyme to form uridine diphosphate glucose (UDPGluc, FIG. No. 2). This can also refer to nitrogen bonds (C—N—C—C) such as those that exist in glycoproteins (see FIG. No. 3), oxygen bonds (C—O—C) as is also the case in certain glycoproteins, or in the polysaccharide structure itself (See FIG. No. 4). In a simple manner this property permits the establishing of covalent bonds between polysaccharides and other polymers (such as, for example, proteins, other polysaccharides, or nucleic acids).

The formation of “nucleotidic sugar” complexes in nature, for example during one of the intermediate steps of the natural synthesis of glycogen as a glucose donor (in this precise case, the sugar-nucleotide bond is a diphosphate bond that results in uridine diphosphate glucose with a glucose-ribose bond, See FIG. No. 2) illustrates this type of interaction and the establishment of covalent bonds between the sugars and nucleic acids. As an example, the polysaccharide-polynucleotide bond can provoke the intervention of links of the type 1→5, sugar1-sugar2, sugar 1 being situated at the end of the polysaccharide, and sugar 2 being that of the nucleotide, situated at the 5′ end of the polynucleotide.

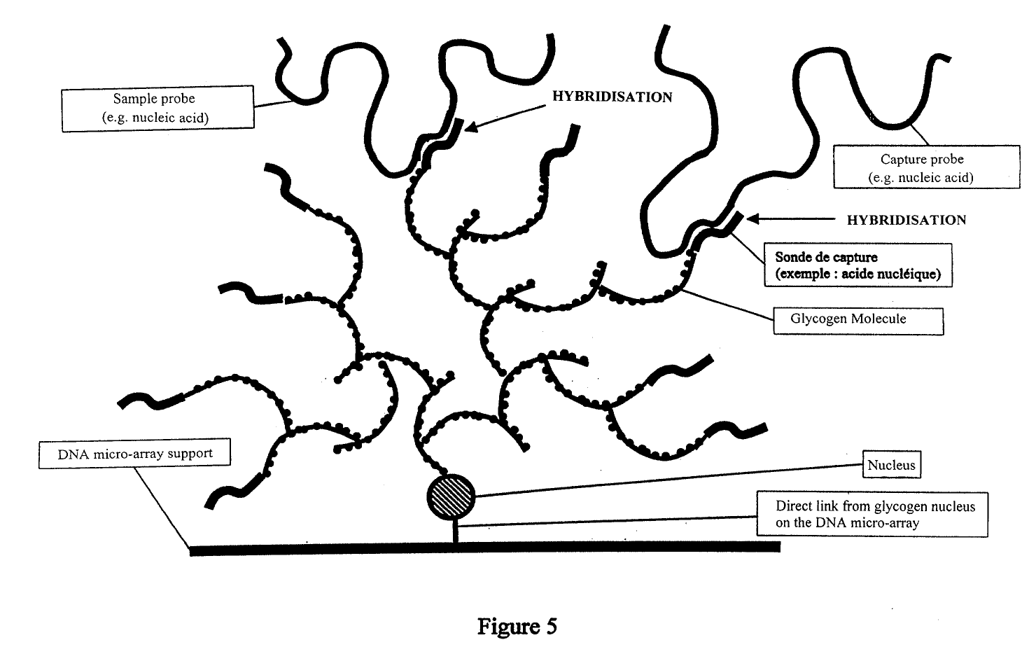

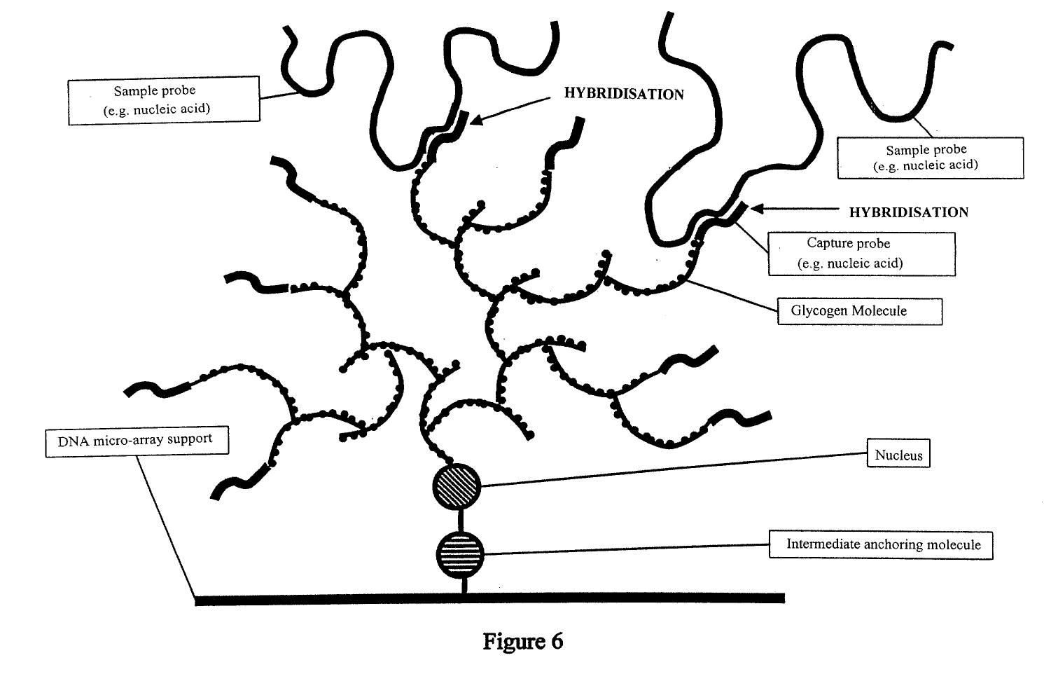

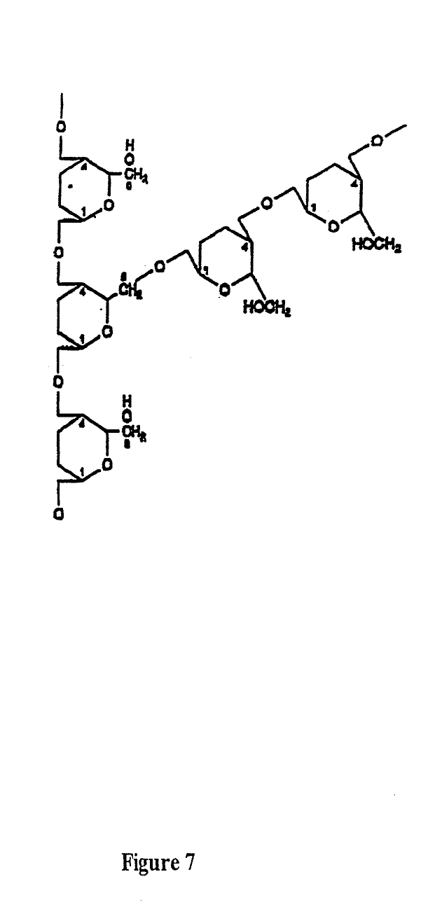

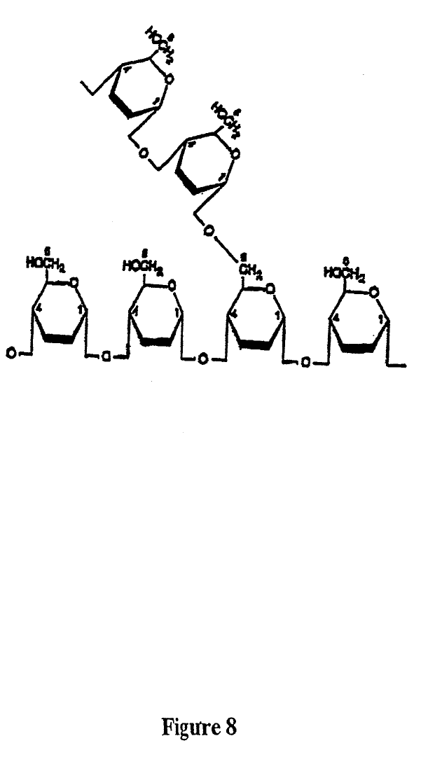

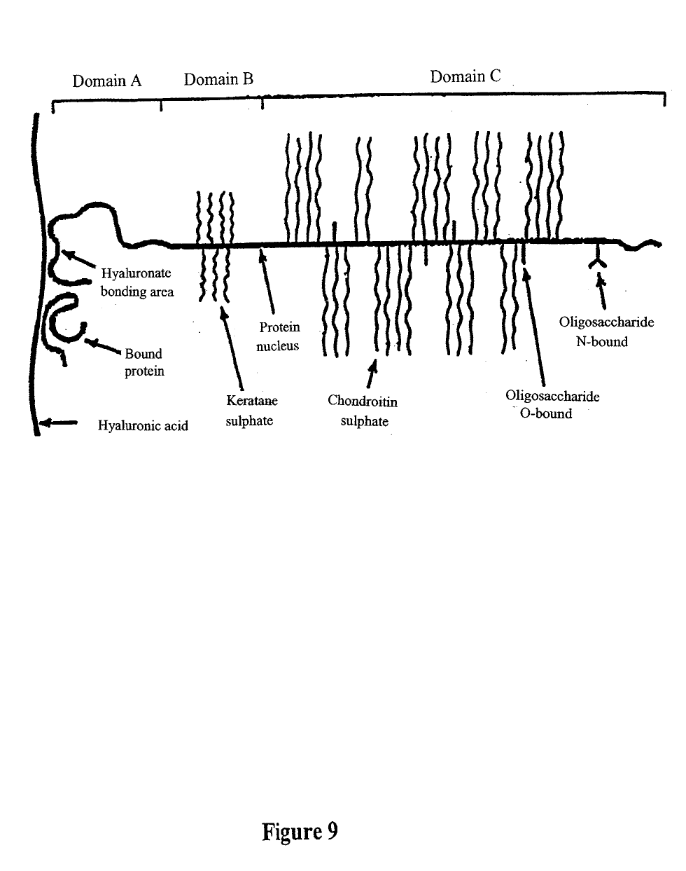

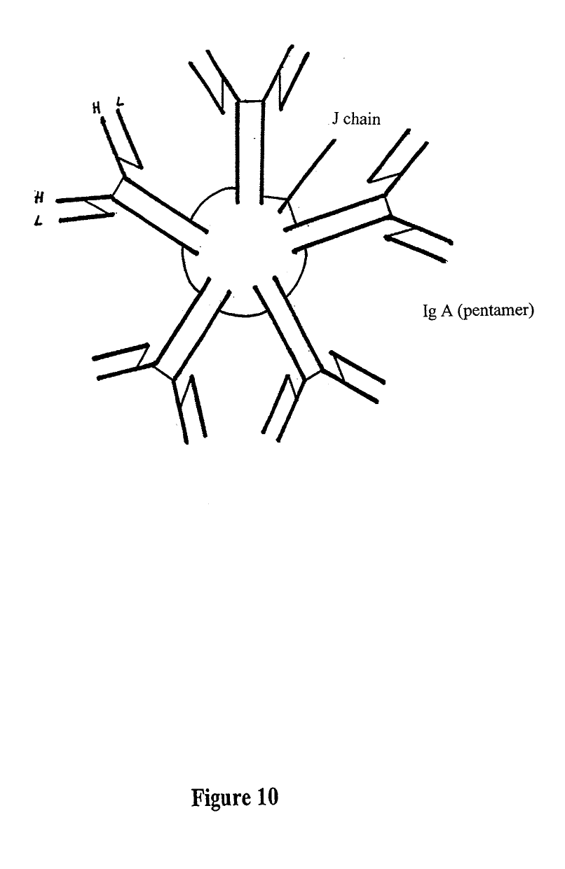

Another advantage of using polysaccharides according to the invention is due to their primary and secondary structure. Indeed, the polysaccharides used in the present invention are multibranched (see FIG. No. 5). In addition, their size (21 nm in diameter for glycogen) is perfectly suited to the miniaturised format of DNA micro-arrays, and the distance between the branchings (approximately 13 glucose residues for natural glycogen) allows advantageously to avoid a too much steric space occupation of the targets fixed on the ends of the branched polymer. A branching on every 2 to 5 residues, would, indeed, hinder probes hybridisation. Moreover, like the glycogen, the polysaccharides have a spherical shape adapted to the surfaces presented by the probes, according to the present invention. (See FIGS. 5 and 6).

Lastly, the use of sugar-based polymers also permits the fixing of sugars to the micro-array support, in a covalent mode, and thanks to one of the chemical bonds such as those described above, either through any one of their ends, or preferably, through their nucleus. This bond can be a direct bond on the support, or an indirect bond through the intermediary of an anchoring molecule, such a molecule being, notably, of proteinic type. Therefore, in the case of natural glycogen, this is linked in covalent mode by its nucleus to a protein, glycogenine (here, by a tyrosine residue). In the case in question, the polysaccharide is therefore used in these applications in the form of a glycoprotein.

A specific example of a molecule of this type is the glycogen molecule, notably endogenic glycogen in the human being. This molecule is a sugar polymer with numerous branchings from a common trunk, these branches also being sugar polymers. A particular aim of the present patent application thus relates to the use of glycogen or glycogen derivatives for this purpose.

Glycogen possesses all the properties cited above. It is commercially available and is inexpensive (See FIG. Nos. 5, 6 and 7: glycogen-polynucleotide array). A glycogen molecule can present more than 60 free glucidic ends for fixing more than 60 probes, representing a very large fixing capacity that is very difficult to obtain through artificial polymers synthesis while maintaining a limited occupied space compatible with the use envisaged in the invention.

Other examples of such biological sugar based compounds are amylopectin, or any other polymer derived from the starch structure (See FIG. No. 8), glycosaminoglycans (in particular muco-polysaccharides), as well as any other polysaccharide, whatever the sugar or sugars contained in the composition (galactose, glucose, mannose, fucose, xylose, N′acetylgalactosamine, N-acetylglucosamine, etc.) that is able to form a branched structure.

The polymer or arborescent organic compound can also be a glycoprotein-based polymer. Such molecules exist in nature, and one of their properties is a structure that has a proteinic nucleus, around which polysaccharides can be covalently linked (O-bonds or N-bonds according to the amino acids that act as residue and on which the polysaccharide chain is fixed) (See FIG. Nos. 3 and 4). These polysaccharides can be fixed in varying numbers, notably in numbers higher than 2. A single glycoprotein can present several polysaccharides with different lengths, with different numbers of residues between branches, and with different chemical nature of the residues (pentoses, hexoses, etc) without this affecting the general principle of their use in these applications. The general structure of these molecules can be branched. In the case in question, the capture probes (DNA, RNA, PNA..) are fixed in covalent mode to the ends of the polysaccharide chains. The glycoprotein bond on the micro-array support can be formed either at the proteinic nucleus level, or at a polysaccharide chain level, but, in this case, preferably at the proteinic nucleus level, and even more preferably, with an O-bond or N-bond with an amino acid residue, either to an intermediate anchoring molecule that is itself directly fixed on the support, or directly to the support.

These molecules possess the properties required for their application in the context of the nucleic acid micro-arrays according to the invention, as is the case with the glycogen-glycogenine complex mentionned previously. In the case of other glycoproteins, the spatial characteristics may vary (molecule size, number of branches, and the number of residues between branches), but they still remain of the same order of magnitude (factor 10) as those cited for glycogen or aggregane and are therefore compatible with their use for nucleic acid micro-arrays.

The present invention also functions with glycoproteins that present several polysaccharide chains in the case in question, where the chains themselves are not branched, but where the external structure of the molecule remains globally spherical, and possesses numerous probes fixing sites in periphery (sea urchin type general structure) and a molecular size similar to that of glycogen or aggregane.

Particular examples of these compounds are proteoglycans, proteins that contain glycosaminoglycans with covalent links. In these molecules, the proteins form the “proteinic nucleus” of the molecule, and the glycosaminoglycans form its branches. Preferably, the invention opts for the use of aggrecane. Aggrecane (See FIG. No. 9) is a glycoprotein present naturally, notably in cartilages. It can exist in its natural form of multiple proteoglycan molecules clustered on a central motif (polymer), such as hyaluronic acid, this cluster being formed through the intermediary of covalent bonds, or not, with the central polymer and leading to the formation of a multi-branched “super-molecule” whose general structural properties are compatible with the invention, and whose free ends, which are sugars, can be linked to targets. Lastly, the proteoglycans are globally charged in negative mode, as this can favour the specificity of the target-probe hybridisation in the case where these are nucleic acids (DNA, RNA).

In another particularrealisation mode, the polymer can also be an amino acids polymer, or any type of molecules with amine groups that react to a nucleophile attack, or other types of chemical groups that can be linked in covalent mode to a nucleic acid.

The organic polymer can therefore be a polymer with proteins or proteinic complex base (composed of several sub-units, also linked with each other by. covalent bonds such as disulphide links) that have a branch type structure.

A particular example of a polypeptidic compound is represented by the immunoglobulins (Ig), whether these are in simple or complex form (as in the case of Type A immunoglobulins, see FIG. No. 10). Since immunoglobulins are natural molecules they are easy to produce in large quantities after selection of adequate clones (monoclonal immunoglobulins). In this application, the immunoglobulins are used as anchoring molecules for the capture probes. The probes are linked to the Fab ends of the immunoglobulins, either with a classic antigen-antibody type bond, in the case where the immunoglobulin is directed against a portion of the probe, or preferably, through an amino acid type covalent bond (of the Ig)—sugar (of the polynucleotide).

Naturally those skilled in the art can select other compounds of biological origin with the characteristics required for use in the present invention, namely, the possibility of creating multiple bonds with a molecule of interest (this can be nucleic acids, for example), an arborescent form that ensures excellent accessibility and that permits an increase in density, as well as an absence of interference with the hybridisation reaction.

To realize the invention, the trunk of the molecule to be used as the fixing arm, is attached to the micro-array support, preferably in covalent mode, and the target nucleic acids are fixed (or directly synthesized) at the ends of several or all of the molecule arborescences, preferably in covalent mode. FIG. 1 shows a spatial representation of nucleic acid micro-array constructed using this kind of arm, compared with micro-arrays constructed using linear arms. Preferably, a single type of arm molecule is used on the same micro-array, at a density that can be adapted by those skilled in the art. However, it is naturally understood that molecules with different structure and/or length and/or shape can be used on the same nucleic acid micro-array.

Nucleic acids can, for example, be fixed to the arm through an amino group that is reactive to a nucleophile attack, whatever the nature of this attack. For example, an N₃ amino group can be linked in covalent mode with a nucleic acid under the action of exposure to light. In a more general manner, the polymer arm preferably contains, on the chain end of its arms, a chemical group, whatever its nature, which can be activated and which has the capacity of being linked in covalent mode with a phosphate group (or other) on the chain end of a nucleic acid. This chemical group with activation capacity can be present during polymer synthesis, or can be added at a later stage. It can be directly active during polymer synthesis or during its attachment on the polymer, or it can be activated chemically at a later stage.

A certain number of chemical modes used to establish covalent bonding between a nucleotide or a nucleic acid on the one hand, and a non-nucleic support, on the other, have been described in prior art, for example for oligonucleotides synthesis in vitro, or for fixing oligonucleotides on a metal support. These techniques can be used in the context of the present invention to fix nucleic acids on a DNA array, notably by fixing them using arms as defined above. This includes the possibility to realize the attachment by means of a system such as the streptavidine type.

Attachment of the arm on the support can be carried out by means of a chemical group with activating capacity, at the end of the trunk, that can interact with support molecules to create a bond, ideally, of the covalent bond type.

As indicated above, the use of arborescent arms according to the present invention offers numerous advantages in terms of target nucleic acids density. For example in the case of “spots” of 20 μm in diameter:

In the case “A” of fixing targets on the support by means of non-arborescent fixing arms (arms/targets stoechiometric molecular ratio =1) or direct fixation of targets on the support, the targets presentation surface (surface on which the probes will have access to become hybridised on the targets) is:

(Π)×(radius)²=(Π)×θ²=α(μm²)

In the case “B” of fixing targets on the support by means of arborescent fixing arms, it can be estimated that each arm forms a hemispherical structure at the targets presentation surface level (fixing surface of the targets on the arm), or, in other terms, that the total target presentation surface is:

(1/2)×(4)×(Π)×(radius)²=2×(Π)×θ²=2×α(μm²)

Therefore this is equivalent to doubling the presentation surface of case “A”, for a constant target density. Or inversely, for the same quantity of targets per “spot”, to reducing the density by a factor 2, and therefore improving the probes access to the targets (reduction of steric occupied space). Extending this principle, if the surface of the arborescent arms is oblong in shape, the presentation surface of the targets is increased in the same manner.

In addition, the fixing density of the targets on the arborescent arms is, in principle, much stronger than that obtained through direct targets fixing on the micro-array support, or by targets fixing using linear arms (non-arborescent) because, for the same number of targets attached per array “spot”, the steric occupied space of the arms themselves on the micro-array support surface is less in the case of arborescent arms compared to linear arms. Therefore, in reality, targets density should be increased by more than a factor 2, according to this principle, or the targets steric occupied space should be reduced by more than a factor 2.

Therefore, this aspect of the invention makes it possible, to increase the targetsdensity per array “spot” on the one hand (and consequently to increase the detection sensitivity for measurement of the probes hybridisation on the targets), and on the other hand, to reduce the steric occupied space of the targets at the same time (and consequently to facilitate probes hybridisation on the targets, that also increases detection sensitivity).

As indicated previously, nucleic acid micro-arrays comprising this type of arm can be composed of a support with a flat and/or convex surface, and be of a solid or semi-solid nature. Moreover, these supports can comprise materials such as glass, silica, poly-lysine, amino-silanes and/or amino-reactive silanes, or negatively charged supports, that will be described in detail below. In addition, the target nucleic acids immobilised on the arm arborescences can also be of varying nature, composition, and origin. They can thus be single or double strand nucleic acids of natural, synthetic and/or semi-synthetic origin. In particular, this can involve synthetic oligonucleotides, PCR products, genes or gene fragments, plasmids (or other vectors such as cosmids, YAC, phages, etc), single or double strand cDNA, or RNA. In addition, the length of the nucleic acids can also vary. However, it is preferable that the nucleic acid lengths be less than approximately 1000 bases (or base pairs).

In a particular embodiment, the target nucleic acids are composed of single strand nucleic acids, notably single strand nucleic acids with a length less than 1000 bases.

In a particular embodiment, the target nucleic acids are constituted of single strand RNA, preferably with a length less than 1000 bases. This can concern notably total RNA or mRNA taken from a biological sample. Indeed, the present invention describes the use of single strand RNA as micro-array targets, for the first time. This constitutes another particular aim of the present patent application.

In another variant of the invention, the method involves single strand DNA, or mixtures of single strand DNA and RNA.

In a preferred embodiment of the present invention, the target single strand nucleic acids have a length between about 25 and 100 nucleotides, preferably between about 30 and 60 nucleotides.

With regard to this aspect, a particular aim of the present invention also features a nucleic acid micro-array composed of single strand nucleic acids fixed on a support, where the single strand nucleic acids have a length between about 25 and 100 nucleotides, preferably between about 30 and 60 nucleotides. The invention also features the use, as target nucleic acids, of a population (of fragments) of single strand nucleic acids with a length between about 30 and 60 nucleotides, designed in the present application by the term “long oligonucleotides”.

According to a first particular variant, the single strand nucleic acids are single strand DNA with a length between about 30 and 60 nucleotides.

According to another particular variant, the single strand nucleic acids are single strand RNA with a length between about 30 and 60 nucleotides.

Advantageously, these are nucleic acids produced by chemical synthesis in vitro, according to techniques familiar to those skilled in the art (synthesizers). At a later stage these oligonucleotides are immobilised on the support.

Contrary to products that are available at the moment, the use (of fragments) of nucleic acids with this length makes it possible to obtain specific probes hybridisation on their corresponding targets on the micro-arrays. Indeed, as described above, the use of oligonucleotides with a length of 25 bases or less, as described in prior art, leads to the presence of numerous non-specific hybridisations of the probes, and consequently, to unsatisfactory reproducibility levels of experiments using this system. On the contrary, the use of longer oligonucleotides, according to the invention, notably with a length between 30 and 60 nucleotides, can obtain absolute hybridisation specificity.

Once they have been synthesized, the long oligo-nucleotides can be fixed on the micro-array support in an ordered manner. Fixing can be either direct (long oligonucleotide fixed directly on the support), or indirect (covalent fixing of the long oligonucleotide on an “arm” as defined and described previously, this arm being fixed to the support). The advantage of fixing the long oligonucleotide indirectly, is the increase in probe accessibility to the oligonucleotide sequence, compared to the direct fixing situation where the steric occupied space is larger. However, direct fixing of long oligonucleotide is possible and can provide sufficient hybridisation specificity for the reproducible use of oligonucleotides arrays. Whatever the fixation mode used, (whether direct or indirect), the oligonucleotides are fixed by one of their ends (5′ or 3′) and not along their total length, in order to provide the probes with sufficient access to the oligonucleotides. Moreover, in the case of indirect fixing, two choices are possible: either the long oligonucleotides are fixed by one of their ends on the arm, and the oligonucleotide-arm complexes are then deposited in an ordered manner on the support and then fixed to the latter, or the arm is attached to the support beforehand, and the long oligonucleotides are then deposited in an ordered manner on the arm-support complex and fixed to the arms.

In this embodiment, the type of support and arm used can be like those described previously. The use of this type of chemically synthesized long oligonucleotide arrays according to the present invention offers certain advantages compared to the use of shorter single strand targets and/or double strand and longer targets produced by PRC:

First of all, the fact that the target is of a single strand nature permits an improvement in the hybridisation reaction yield of the probe on the target. Indeed, if the target is double stranded, a large number of the targets will hybridise on themselves during this reaction (the second strand of the target is in competition with the probe for hybridisation).

Then, direct chemical synthesis of targets is much simpler to manage than PCR target synthesis. In the first case, it is only necessary to know the sequences of the targets to be deposited on the micro-array, whereas, in the second case, the nucleic acids containing these sequences must be deposited materially (generally in the form of clones) in order to perform specific amplification through PCR.

Moreover, amplification using PCR produces a heterogeneous double strand nucleic acids population with the presence of contaminating fragments in the majority of cases, even though these fragments are in a minority in the final reaction product. This leads to the presence of non-specific probe-target hybridisations, and consequently, background noise radiation during hybridisation rates measurement. On the contrary, direct single strand targets synthesis makes it possible to obtain very homogeneous nucleic acid sequence fragments, and at termination, to obtain targets that are more pure and consequently with less non-specific probe-target hybridisation. This specificity is guaranteed even more when the micro-arrays include arms, arborescent or not, and are electrically charged as will be described below.

Lastly, chemical synthesis of single strand probes can be automated in order to obtain purified targets, whereas double strand targets produced through PCR must be purified before they are fixed on the support.

According to the present invention, long synthetic oligonucleotide arrays represent a significant technical advantage compared to products and methods described in prior art. This advantage is even greater when the nucleic acid micro-arrays include arborescent arms or electrically charged arms or supports.

In fact, the present invention also describes new approaches for controlling the spatial arrangement of nucleic acids and/or of arms on the nucleic acid micro-array in order to facilitate presentation conditions, and consequently, the probe hybridisation on the targets. In particular, the present invention can now demonstrate that it is possible to determine (and to modify) the electrical characteristics of the arms (whether these are organic polymers of proteinic, glucidic, or lipidic nature, or non organic synthesis polymers) or micro-array supports in order to improve hybridisation conditions, notably, micro-arrays selectivity and sensitivity.

This aspect of the invention can be applied to all possible natures of oligonucleotide fixing arms on array supports, whether their primary structure is linear or arborescent, and whatever the nature of the micro-array support.

In fact, it is well-known that nucleic acids are negatively charged (because of the fact that they are weak acids that, under normal conditions, are in environments with a pH higher than their pKa; their acid functions are situated on the phosphate groups that ensure the bonding between the nucleotide sugars). Interaction between nucleic acid sequences can be summed up, in a simplified manner, as follows: on the one hand, these sequences can be hybridised between each other by establishing hydrogen bonds and Van der Waals attraction between the complementary bases (that could be considered as a common affinity), and on the other hand, they can repel each other because of the negative charges at the phosphate group level. To obtain stable hybridisation between two single strand nucleic acid molecules, the attraction linked with the hydrogen and the Van der Waals bonds, must be stronger than the repulsion linked with the negative charges, which is the case when the number of complementary adjacent bases is sufficient to obtain an attraction that is stronger than the repulsion. Another parameter to be taken into consideration is that of the atomic stresses of molecular folding-in (secondary and tertiary structures). These general principles concerning nucleic acids are just as valid within any single strand nucleic acid molecule, whatever its length. They provide the possibility of explaining the capacity or lack of capacity of such a certain molecule to hybridise itself or not. As far as the target fixing arms onto the micro-array support are concerned, these are also subject to secondary structure and steric occupied space atomic stresses, but they do not present any attraction or hybridisation phenomenon such as those described for nucleic acids. These stresses on the arms are also influenced by the fact that the nucleic acid strands are fixed to their end(s).

One of the problems posed by the creation of micro-arrays, whether the fixed targets are single strand oligonucleotides (whatever their length) or double strand DNA (whether this concerns entire plasmids or polymer chain reaction products) (PCR), is the accessibility of these targets to the probe molecules that are to be analysed and hybridised in a specific manner on their corresponding targets.

This accessibility conditions the hybridisation reaction yield, and consequently, the sensitivity and specificity of the probe analysis. This probe-target hybridisation accessibility is reduced notably by the steric occupied space of the targets fixed on the micro-array, by the interactions between targets located in proximity to each other because of the steric occupied space (partial hybridisations) and by their secondary structures (intra-molecular folding-in of the targets). The problems linked with the steric occupied space are generated by the need to deposit targets with high density on each array “spot” in order to be able to detect weak specific nucleic acid rates (detection sensitivity) and reduce the effects of hybridisation capacity saturation that can hinder the quantification of hybridisation signals. This is the reason why a general principle consists of depositing molecular quantities of targets in excessive number, compared to the corresponding probe molecules.

The present invention provides a solution to these problems. In fact, it describes the use of special arms or supports, reducing the ionic interferences between the targets, thus ensuring better hybridisation, including those cases where the targets have high-density levels. More particularly, the present application demonstrates that to fix the targets on the micro-array supports, it is possible to use fixing arms with a negative electrical charge or negatively charged supports.

Therefore, another aim of the invention concerns a micro-array comprising nucleic acids immobilised on a support by means of arms carrying a negative electrical charge. Another aim of the present invention concerns a nucleic acid micro-array comprising nucleic acids immobilised on a support carrying a uniform negative electrical charge.

The present invention also features the use of negatively charged molecules or negatively charged supports for the preparation of nucleic acid micro-arrays. The use of fixing arms that are negatively charged in a uniform manner in experimental conditions of probe hybridisation on the targets advantageously provides the following phenomena:

the arms exert electrical repulsive forces against the adjacent arms resulting in reducing their folding in on themselves and their steric occupied space at the same time, notably at the level of their ends fixed on the targets;

the arms exert electrical repulsive forces against the targets fixed at their ends, resulting in pushing the targets towards arranging themselves outwards—that is: moving away from the micro-array support, making them more accessible to the probes;

these mutual repulsive forces result in limiting the attractive interactions between adjacent probes by moving them away from each other, making them more accessible to the probes, and reducing the risks of mutual attraction linked with the Van der Waals forces or hydrogen bonds,

this negative electrical field exercised next to the targets fixed to the arms tends to dis-assemble the secondary target structures (in other words—it aligns them) and consequently it reduces the folding in of the targets on themselves, making them more accessible to the probes;

the layer located closest to the micro-array support and formed by the arms is negatively charged globally, causing it to exercise an electrical repulsive force against probes (also negatively charged) and therefore, to reduce the non-specific hybridisation phenomena of the probe-targets. This also helps to reduce the background noise phenomenon during hybridisation result analysis, and consequently, to increase the sensitivity and specificity of the analyses by DNA micro-array technology at the same time.

This particular aspect of the invention thus provides a clear technological improvement to the methods of the art, ensuing better micro-array selectivity and sensitivity.

Naturally, this aspect of the invention is not limited to arrays where arms are used. On the contrary, it is also applicable for the use of a micro-array support negatively charged in a uniform manner, in those situations where the targets are directly fixed onto the support without intermediate arms. This method is valid whether the negative charges on the support are applied previously, or after the targets have been attached on the support. This is a different principle from that which consists in depositing targets on micro-electrodes to which an electrical current can be applied, because in the latter case the electrical charge on the support is not homogeneous.

In this aspect of the invention, the micro-arrays can be composed of all the different types of target nucleic acid populations as defined above, and the negatively charged arms can also be composed of an arborescent form as mentioned above.

The present invention also concerns a micro-array preparation process, as described above, comprising the immobilisation (or fixing) of nucleic acids on a support by means of arborescent shaped arms comprising a polymer of biological origin.

The preparation process can comprise an initial fixing step of the arborescent shaped arm on the support, followed by a second fixing step of the target molecule to the arborescent arm. According to another embodiment of the present invention, the nucleic acid micro-array preparation process can comprise an initial step where the target molecule is fixed on the arborescent shaped arm, followed by a second step where the complex, obtained during the initial step, is fixed onto the support.

The present invention also relates to the use of nucleic acid micro-arrays as described above, for an experimental, therapeutic, diagnosis purpose. In particular, the invention concerns the use of the micro-arrays described above for:

studying the regulation of genetic expression, or

research of genes or gene fragments, or

target identification, or

genetic diagnosis.

In these applications, the micro-arrays are placed in contact with a “probes” nucleic acid population, generally marked previously, then the hybridisation profile of the probes on the nucleic acid micro-array is determined according to the techniques familiar to those skilled in the art. Thus, one aim of the invention also concerns a process for polynucleotide analysis, comprising the placing of a population of polynucleotides, preferably marked, in contact with a nucleic acid micro-array according to the invention, and the demonstration of hybrid formation.

Naturally, it is understood that the present invention is not limited to the specific production methods described above, but also covers the execution variants included in the normal know-how of those skilled in the art.

1. Micro-array characterised in that it comprises nucleic acids immobilised on a support by means of arborescent shaped arms, comprising a polymer of biological origin.

2. Micro-array according to claim 1, characterised in that the arm is a branched polymer with one or several branching levels.

3. Micro-array according to claim 1 or 2, characterised in that the arm is an organic polymer.

4. Micro-array according to any one of claims 1 to 3, characterised in that the arm is a sugar polymer, for example glycogen or a glycogen derivative or amylopectin.

5. Micro-array according to any one of claims 1 to 3, characterised in that the arm is a branched polymer comprising repeated galactose, glucose, mannose, fucose, xylose, N-acetylgalactosamine and/or N-acetylglucosamine monomers.

6. Micro-array according to any one of claims 1 to 3, characterised in that the arm is a glycopolypeptide, for example aggrecane.

7. Micro-array according to any one of claims 1 to 3, characterised in that the arm is a polypeptide compound, for example an immunoglobulin.

8. Micro-array according to any one of the preceding claims, characterised in that the nucleic acids are fixed in covalent mode to the ends of the arborescent arms.

9. Micro-array according to any one of the preceding claims, characterised in that the arms are fixed in covalent mode to the support by the trunk part of the molecule.

10. Micro-array according to any one of the preceding claims, characterised in that the support has a flat and/or convex surface.

11. Micro-array according to claim 10, characterised in that the support is a solid or semi-solid support.

12. Micro-array according to claim 11, characterised in that the support is composed of glass, silica, poly-lysine, amino-silanes and/or amino-reactive silanes.

13. Micro-array according to any one of the preceding claims, characterised in that the nucleic acids are single or double strand nucleic acids, of natural, synthetic and/or semi-synthetic origin.

14. Micro-array according to claim 13, characterised in that the nucleic acids are chosen among synthesized oligonucleotides, PCR products, genes or gene fragments, plasmids, single or double strand cDNA or RNA.

15. Micro-array according to claim 14, characterised in that the nucleic acids are single strand nucleic acids, notably molecules of single strand nucleic acids with a length between approximately 25 and 100 nucleotides, preferably between approximately 30 and 60 nucleotides.

16. Use of a polymer of biological origin (notably a sugar polymer, and in particular derived from glycogen) with a spatial organisation in arborescent form, for fixing nucleic acids on supports.

17. Micro-array according to claim 13, characterised in that the nucleic acids are single strand nucleic acids with a length of approximately 30 to 60 nucleotides.

18. Micro-array according to claim 17, characterised in that the single strand nucleic acids are single strand DNAs with a length of approximately 30 to 60 nucleotides.

19. Micro-array according to claim 17, characterised in that the single strand nucleic acids are single strand RNAs with a length of approximately 30 to 60 nucleotides.

20. Micro-array according to any one of claims 17 to 19, characterised in that the single strand nucleic acids are nucleic acids produced by chemical synthesis in vitro.

21. Micro-array according to any one of claims 17 to 20, characterised in that it comprises RNAs immobilised on a solid or semi-solid support by means of arborescent form arms comprising a polymer of biological origin.

22. Micro-array according to any one of claims 17 to 20, characterised in that it comprises nucleic acids produced by PCR immobilised on a solid or semi-solid support by means of arborescent form arms comprising a polymer of biological origin.

23. Micro-array according to any one of claims 1 to 15 and 17 to 22, characterised in that the arm carries a negative electrical charge.

24. Micro-array according to any one of claims 1 to 15 and 17 to 22, characterised in that the support carries a uniform negative electrical charge.

25. Use of a micro-array according to any one of claims 1 to 15 and 17 to 24, for studying the regulation of genetic expression.

26. Use of a micro-array according to any one of claims 1 to 15 and 17 to 24, for research of genes or gene fragments.

27. Use of a micro-array according to any one of claims 1 to 15 and 17 to 24, for targets identification.

28. Use of a micro-array according to any one of claims 1 to 15 and 17 to 24, for genetic diagnosis.

29. Process for micro-array preparation according to any one of claims 1 to 15 and 17 to 24, characterised in that it comprises:
a) a step for fixing the arborescent form arm on the support, and
b) a step for fixing the target molecule to the arm in arborescent form obtained during a).

30. Process for micro-array preparation according to any one of claims 1 to 15 and 17 to 24, characterised in that it comprises:
a) a step for fixing the target molecule to the arm in arborescent form,
b) a step for fixing the complex obtained during a) on the support.
